(19) United States
(12) Patent Application Publication
(10) Pub. No.: US 2009/0076322 A1
(43) Pub. Date: Mar. 19, 2009

(54) CAPSULE ENDOSCOPE

(76) Inventors: Atsushi MATSUNAGA, Kanagawa (JP); Masayuki HAYASHI, Kanagawa (JP); Masaya NAKAYAMA, Kanagawa (JP)

Correspondence Address: BIRCH STEWART KOLASCH & BIRCH, PO BOX 747, FALLS CHURCH, VA, 22040-0747, US

(21) Appl. No.: 12/204,694

(22) Filed: Sep. 4, 2008

(30) Foreign Application Priority Data
Sep. 13, 2007 (JP) 2007-238258
Apr. 30, 2008 (JP) 2008-119002

(51) Int. Cl. A61B 1/04

(52) U.S. Cl. 600/109

(57) ABSTRACT

A capsule endoscope includes at least an imaging device that images a location of a subject and an optical system that focuses the location at the imaging device. The imaging device has plural pixel portions that are arranged in in-plane directions on a substrate. The pixel portion has a photoelectric conversion portion that includes a lower electrode, a photoelectric conversion layer formed over the lower electrode, and an upper electrode formed over the photoelectric conversion layer, and a signal output portion that outputs a signal based on a charge generated at the photoelectric conversion layer through a field effect thin film transistor. The field effect thin film transistor includes at least a gate electrode, a gate insulation film, a semiconductor layer, a source electrode and a drain electrode. The photoelectric conversion portion and the signal output portion are superposed in plan view.

### CROSS-REFERENCE TO RELATED APPLICATION

This application claims priority under 35 USC 119 from Japanese Patent Applications No. 2007-238258 and No. 2008-119002, the disclosures of which are incorporated by reference herein.

### BACKGROUND OF THE INVENTION

1. Technical Field

The present invention relates to a capsule endoscope.

2. Related Art

A capsule endoscope, in which an imaging device or the like is incorporated into a capsule, is swallowed by a patient and hence captures images of digestive organs and the like. Such a capsule endoscope reduces impositions on the patient compared to a conventional type of endoscope, for which a tube is inserted.

As imaging devices incorporated in capsule endoscopes, CMOS-type (complementary metal oxide semiconductor) imaging devices (below referred to as CMOS sensors) are known.

A CMOS sensor is formed by, for example, arraying embedded-type photodiodes (photoelectric conversion portions), which generate charges, and signal output portions, which are structured with ring-form gate electrodes or the like, in in-plane directions on a substrate. Further, as illustrated in Japanese Patent Application Laid-Open (JP-A) No. 2007-105236, a light-blocking film is provided over the photodiodes (photoelectric conversion portions) and the signal output portions, and apertures are formed in the light-blocking film at positions corresponding to the photodiodes (photoelectric conversion portions).

### SUMMARY

Reductions in size of capsule endoscopes are desired, in order to, for example, make them easier for patients to swallow and suchlike.

In consideration of the circumstances described above, the present invention will reduce the size of a capsule endoscope.

A capsule endoscope of a first aspect of the present invention includes at least an imaging device that images a target location of a subject and an optical system that focuses the target location at the imaging device. The imaging device has plural pixel portions that are arranged in in-plane directions on a substrate. The each pixel portion includes a photoelectric conversion portion that includes a lower electrode, a photoelectric conversion layer formed over the lower electrode, and an upper electrode formed over the photoelectric conversion layer; and a signal output portion that outputs a signal based on a charge generated at the photoelectric conversion layer through a field effect thin film transistor. The field effect thin film transistor includes at least a gate electrode, a gate insulation film, a semiconductor layer, a source electrode and a drain electrode. The photoelectric conversion portion and the signal output portion are superposed in plan view.

### BRIEF DESCRIPTION OF THE DRAWINGS

Embodiments of the present invention will be described in detail based on the following figures, wherein:

FIG. 1 is a schematic plan view showing an example of an arrangement of primary light detection pixels of an imaging device provided in a capsule endoscope relating to the present invention;

FIG. 2 is a schematic sectional view showing an example of a layer structure of secondary light detection pixels which constitute the primary light detection pixels;

FIG. 3 is a schematic view specifically illustrating an example of structure of a first secondary light detection pixel;

FIG. 4 is a diagram showing an example of circuit structure of a thin film transistor included in a first layer secondary light detection pixel;

FIG. 5 is a schematic sectional view showing an example of a thin film transistor (a bottom-gate type) in which a semiconductor layer has a two-layer structure;

FIG. 6 is a schematic sectional view showing another example of a thin film transistor (a top-gate type) in which a semiconductor layer has a two-layer structure;

FIG. 7A is a schematic view illustrating general structure of a capsule endoscope apparatus provided with the capsule endoscope relating to the present invention;

FIG. 7B is a view showing a computer which serves as an example of a display device for displaying captured images which are imaged by the capsule endoscope; and

FIG. 8 is a diagram schematically showing general structure of the capsule endoscope relating to the present invention.

### DETAILED DESCRIPTION OF THE INVENTION

Herebelow, an example of an exemplary embodiment of the capsule endoscope of the present invention will be described in detail.

Firstly, schematics of overall structure of a capsule endoscope apparatus 500 will be described.

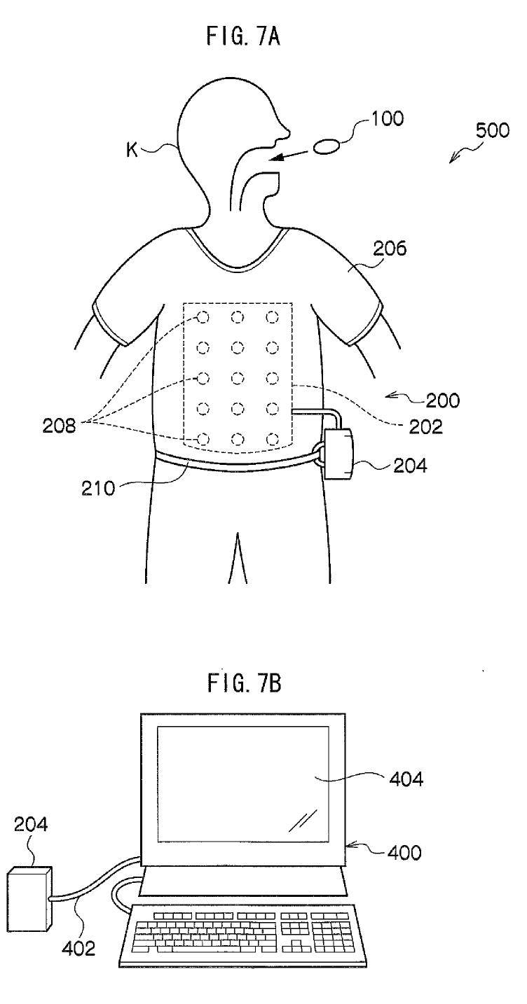

As shown in FIG. 7A, the capsule endoscope apparatus 500, which performs endoscopic examinations, is constituted with a capsule endoscope 100 relating to the exemplary embodiment of the present invention and an external apparatus 200.

The capsule endoscope 100 is swallowed through the mouth of a patient K, and hence, while passing through tubes in a body cavity within the living body, images subjects which are inner wall faces of the alimentary canal in the body (the stomach, small intestine, large intestine and the like), and transmits captured image signals by wireless.

Meanwhile, at the external apparatus 200, an antenna unit 202 disposed outside the body of the patient K receives the image signals transmitted from the capsule endoscope 100, and an image processing unit 204 applies predetermined image processing and saves image data. During examination or after examination has finished, the image data accumulated at the image processing unit 204 of the external apparatus 200 is connected to a computer 400, which serves as a display device shown in FIG. 7B, by a cable 402 or the like, and captured images are displayed at a monitor 404.

The images saved at the image processing unit 204 are stored to a hard disk inside the computer 400, and the images can be displayed at the monitor 404 after, for example, image analysis or the like is performed. Rather than the general purpose computer 400, a dedicated display device (display system) may be used.

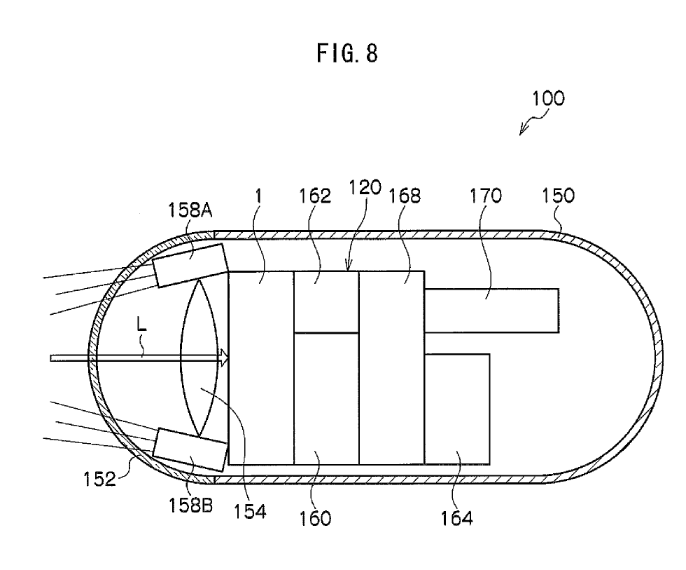

As shown in FIG. 7A, the antenna unit 202 of the present exemplary embodiment is structured with plural antennae 208 mounted at the inner side of a shield chassis 206 with a shielding function, which is worn by the patient K. The image signals which have been captured by the capsule endoscope 100 and transmitted from a built-in antenna 170 (see FIG. 8) are received by the antenna unit 202.

The image processing unit 204 is formed in a box shape, and is provided, at a side face, with a monitor (not shown) which serves as a display device that performs image display and with control buttons (not shown) which implement control functions, or the like. Inside the image processing unit 204, a transmission/reception circuit (communications circuit), a control circuit, an image data display circuit, a power supply and the like are provided.

As mentioned above, the antenna unit 202 is structured with the plural antennae 208 being mounted at the inner side of the shield chassis 206 that has a shielding function, which is worn by the patient K. The image processing unit 204 can be removably attached to a belt 210 of the patient K or the like. Thus, when the capsule endoscope 100 is swallowed and is imaging within the body (i.e., during an examination), the patient K is able to move substantially freely.

Next, the capsule endoscope 100 relating to the present exemplary embodiment will be described.

As shown in FIG. 8, the capsule endoscope 100 is enclosed by a capsule 150. One end of the capsule 150 has a hemispherical form, and at the other end, which is opened, a hemispherical-form transparent cap 152 is attached. White LEDs 158A and 158B, a lens 154, an imaging device 1, a control section 120 and suchlike are also incorporated into the capsule endoscope 100 (capsule 150).

The two white LEDs 158A and 158B are disposed at the inner side of the transparent cap 152, and the lens 154 is mounted between the two white LEDs 158A and 158B. The imaging device 1 (which will be described in detail later), which is capable of capturing color images, is provided at a focusing position of the lens 154. Here, arrow L shows incident light entering the imaging device 1.

The control section 120, which administers overall operations of the capsule endoscope 100, is provided at a rear face side of the imaging device 1. The control section 120 is provided with a processing section 160 and a memory 162, the processing section 160 implements driving of the white LEDs 158A and 158B and the imaging device 1, processing of image signals captured by the imaging device 1 and the like, and the memory 162 memorizes image data that has been captured by the imaging device 1.

A transmission/reception section 168, which transmits and receives radio waves to and from the external apparatus 200 (see FIG. 7A) is provided at the rear face side of the memory 162 and the processing section 160 (i.e., of the control section 120). The antenna 170 is connected to the transmission/reception section 168.

A battery 164, which serves as a power supply, is provided inside the capsule 150. The battery 164 is electrically connected to respective structural components of the above-mentioned white LEDs 158A and 158B, imaging device 1, processing section 160, memory 162, transmission/reception section 168 and the like, and provides electrical power to the respective structural components as required.

At this capsule endoscope 100, light emitted from the white LEDs 158A and 158B passes through the transparent cap 152 and is reflected by the interior of the body, and the reflected light, as incident light L, passes through the transparent cap 152 and the lens 154 and is incident on the imaging device 1. Then, after photoelectric conversion by the imaging device 1, signals obtained by the photoelectric conversion are inputted to the control section 120, and image signals outputted from the control section 120 are transmitted from the antenna 170 of the transmission/reception section 168 to the antennae 208 of the antenna unit 202 of the external apparatus 200 (see FIG. 7A).

In the present exemplary embodiment, the lens 154 alone is employed as an optical system for focusing the incident light L onto the imaging device 1, but this is not a limitation. For example, in addition to the lens 154, a mechanism for focus point adjustment and a mechanism for zooming or the like may be provided, or there may be a mechanism for focus point adjustment and zooming, or the like.

Further, in the present exemplary embodiment, the white LEDs 158A and 158B are employed as an illumination unit for illuminating an imaging region of a subject, but this is not a limitation. For example, other light-emitting bodies such as miniature light bulbs, organic electroluminescents and the like may be employed.

Further still, in the present exemplary embodiment, the battery 164 which is structured as a primary cell is employed as the power supply, but this is not a limitation. For example, another battery (power supply) such as a secondary cell or the like may be employed.

Next, the imaging device 1 will be described.

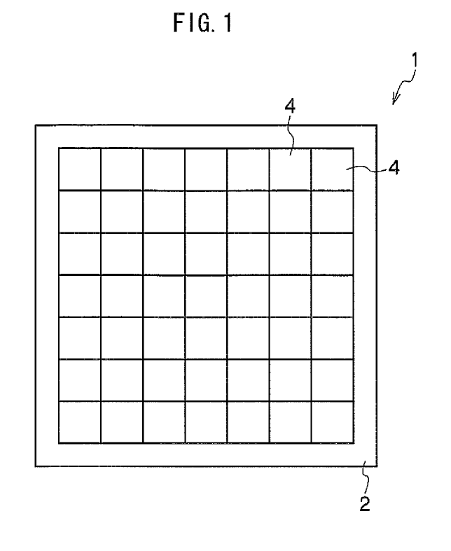

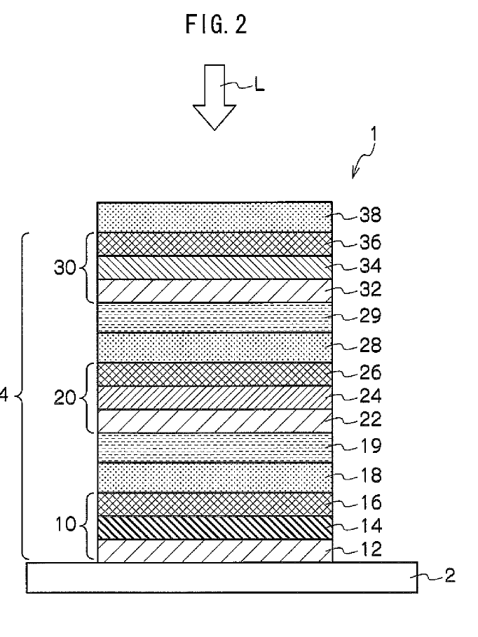

FIG. 1 is a schematic plan view showing an example of an array of primary light detection pixels 4 in directions in the plane of a substrate 2 of the imaging device 1. FIG. 2 is a schematic sectional view showing an example of a layer structure of secondary light detection pixels 10, 20 and 30, which constitute the primary light detection pixels 4. The arrow L shows incident light (see FIG. 8).

As shown in FIG. 2, as pixel portions in the imaging device 1, the three kinds of secondary light detection pixels (light-receiving pixels) 10, 20 and 30, which selectively sense light of respectively different wavelength regions (R, G and B) are layered in a thickness direction at one side (one face) of the substrate 2. Between the neighboring secondary light detection pixels 10, 20 and 30, respective sealing insulation films 18 and 28 and smoothing layers 19 and 29 are interposed.

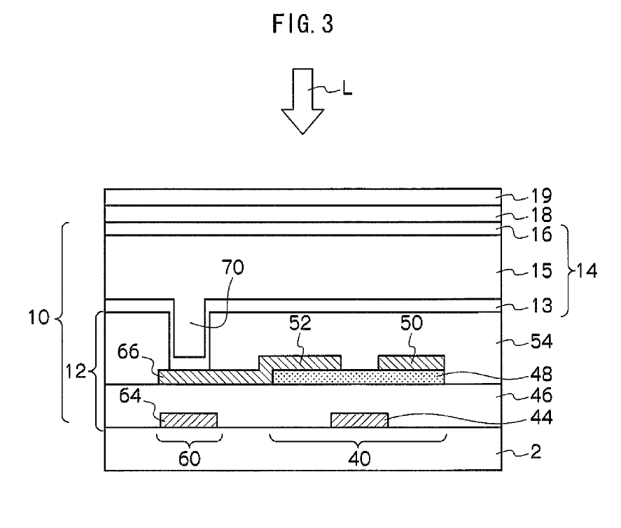

FIG. 3 more specifically illustrates structure of the first secondary light detection pixel 10, which is formed on the substrate 2 first. The first secondary light detection pixel 10 is structured to include a photoelectric conversion portion 14, which selectively senses and photoelectrically converts light of a particular wavelength region, and a signal output section 12, which outputs a signal in accordance with a charge generated by the photoelectric conversion portion 14, through a field effect thin film transistor 40. The field effect thin film transistor 40 includes a gate electrode 44, a gate insulation film 46, a semiconductor layer 48, a source electrode 50 and a drain electrode 52, and the semiconductor layer 48 is formed of an oxide semiconductor or an organic semiconductor (detailed structure of the field effect thin film transistor 40 will be described later).

The second secondary light detection pixel 20 and the third secondary light detection pixel 30 have similar structures, so will not be illustrated or described. That is, the second and third secondary light detection pixels 20 and 30 have the same structure as the first secondary light detection pixel 10, except in using materials that sense light of different wavelength regions in respective photoelectric conversion portions 24 and 34. Accordingly, for the field effect thin film transistor 40 and the like that are included in the first secondary light detection pixel 10, similar field effect thin film transistors and the like are also included in the second and third secondary light detection pixels 20 and 30.

On the substrate 2, the secondary light detection pixels 10, 20 and 30, which sense light corresponding to red (R), green (G) and blue (B), respectively, are layered with the sealing insulation films 18 and 28 interposed, and thus constitute the primary light detection pixels 4. As shown in FIG. 1, the primary light detection pixels 4 are arranged in, for example, a matrix pattern in planar directions of the substrate 2.

The arrangement (array) of the primary light detection pixels 4 is effective for improving resolution when the primary light detection pixels 4 are arrayed in a matrix on the substrate 2 as shown in FIG. 1, but is not limited thus. Suitable settings in accordance with a required resolution and the like are possible. Furthermore, sizes, numbers and the like of the primary light detection pixels 4 may be determined in accordance with required resolution, which may be, for example, 200 ppi or more.

Next, the photoelectric conversion portion 14 will be described.

As shown in FIG. 3, at the photoelectric conversion portion 14, a photoelectric conversion layer 15 is formed between a lower electrode (pixel electrode) 13 and an upper electrode (counter electrode) 16.

The photoelectric conversion layer 15 is structured of an organic material (which will be described in detail later), and is structured such that the photoelectric conversion layers of the layered three kinds of secondary light detection pixels 10, 20 and 30 (see FIG. 2) sense and photoelectrically convert lights of respectively different wavelength regions.

In the present exemplary embodiment, the secondary light detection pixels 10, 20 and 30 are structured so as to selectively absorb and photoelectrically convert, of the incident light L, blue light (for example, wavelengths from 400 nm to 500 nm), green light (for example, wavelengths from 500 nm to 600 nm) and red light (for example, wavelengths from 600 nm to 700 nm), respectively.

That is, the third secondary light detection pixel 30 is structured so as to absorb and photoelectrically convert red light, and permeate green and blue light. The second secondary light detection pixel 20 is structured so as to absorb and photoelectrically convert green light and permeate blue and red light. However, because light in the red wavelength region is absorbed by the third secondary light detection pixel 30 and does not reach the second secondary light detection pixel 20, the second secondary light detection pixel 20 may just as well absorb green light and red light. The first secondary light detection pixel 10 is formed so as to absorb and photoelectrically convert at least blue light. That is, because red light and green light have already been absorbed by the third and second third secondary light detection pixels 30 and 20, and do not reach the first secondary light detection pixel 10, the first secondary light detection pixel 10 may just as well absorb light of all three primary colors.

The secondary light detection pixels 10, 20 and 30 have structures in which the signal output section 12 and the photoelectric conversion portion 14 are layered in a vertical direction (thickness direction) in sectional view. Therefore, in plan view (viewing from the incidence direction of the incident light L), the signal output section 12 and the photoelectric conversion layer 15 have a superposed (overlapping) structure. In the present exemplary embodiment, in plan view, the signal output section 12 is superposed so as to be provided within a region of the photoelectric conversion portion 14 (i.e., in plan view, the signal output section 12 does not protrude from the region of the photoelectric conversion portion 14).

Next, the field effect thin film transistor (TFT, same as hereinbelow) 40, of the signal output section 12 will be described.

As shown in FIG. 3, the secondary light detection pixel 10 outputs a signal based on a charge produced by the photoelectric conversion portion 14 through the signal output section 12. The signal output section 12 is structured to include a capacitor 60 and the field effect thin film transistor 40. The field effect thin film transistor 40 is formed with the gate electrode 44, the gate insulation film 46, the semiconductor layer 48, the source electrode 50 and the drain electrode 52, and the semiconductor layer 48 is formed of an oxide semiconductor or an organic semiconductor. The second and third secondary light detection pixels 20 and 30 are provided with signal output sections 22 and 32 that include field effect thin film transistors with respectively similar structures, and output signals based on charges produced by the respective photoelectric conversion portions 24 and 34.

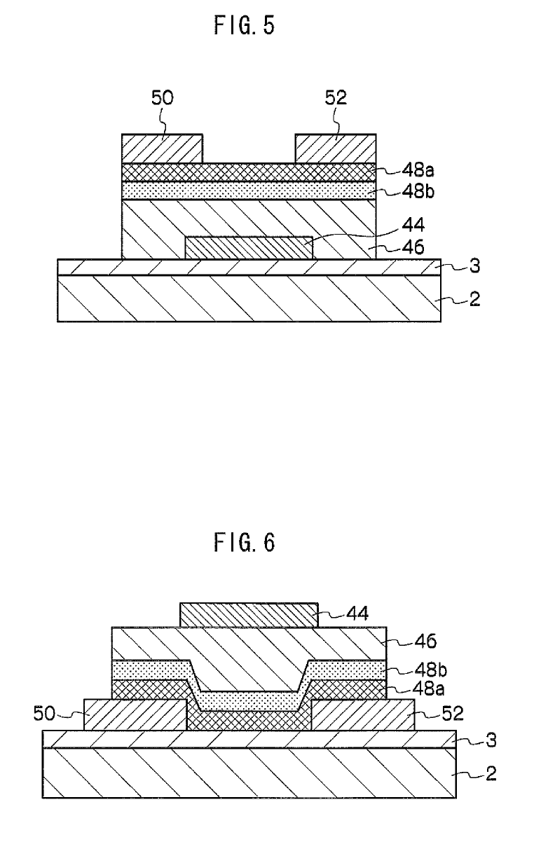

In the present exemplary embodiment, as shown in FIG. 5 and FIG. 6, at the field effect thin film transistor 40, the semiconductor layer 48 includes at least a resistance layer 48a and an active layer 48b with a greater electrical conductivity than the resistance layer 48a. The active layer 48b is in contact with the gate insulation film 46, and the resistance layer 48a is structured to electronically connect the active layer 48b with at least one of the source electrode 50 and the drain electrode 52 (this will be described in more detail later).

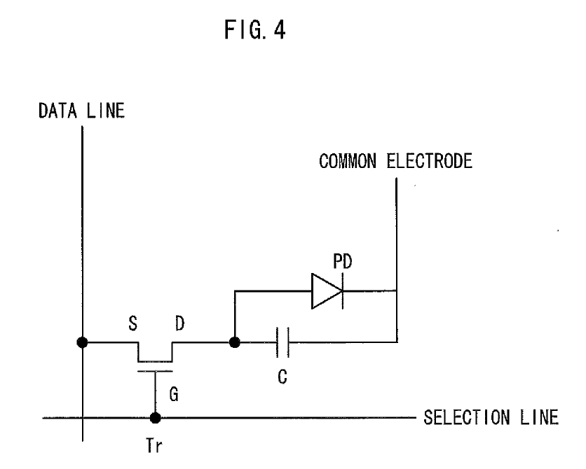

FIG. 4 schematically shows an example of a circuit that is provided at one layer of the secondary light detection pixels in one of the primary light detection pixels 4. Firstly, a gate electrode G of a field effect thin film transistor Tr is selected via a selection line, and a reverse bias voltage required for photoelectric conversion is supplied to a photodiode PD. In this state, when light of a particular wavelength region within incident light from the substrate 2 side is received, a photoelectric current is generated in the photodiode PD. This signal is read through the data line, amplified by an amplifier, subjected to analog signal processing, A-D converted, and subjected to digital signal processing.

It is sufficient to form at least one of the field effect thin film transistor Tr in one of the secondary light detection pixels, but two or more may be provided. An arrangement of the field effect thin film transistor Tr and the capacitor C is not limited to the arrangement shown in FIG. 4, and may be suitably designed. In any case, however, it will be desirable to form the semiconductor layer 48 of an oxide semiconductor or an organic semiconductor.

In a case in which the semiconductor layer 48 is an oxide semiconductor, the oxide semiconductor may be any of monocrystalline, polycrystalline, microcrystalline and non-crystalline, but non-crystalline is desirable. Moreover, an oxide semiconductor is preferable that includes at least one of Cu, Ag, Au, Zn, Cd, Hg, Ga, In, Ti, Ge, Sn, Pb, As, Sb and Bi, and In, Ga, Zn and Sn are more preferable.

Next, output of signals of R (red), G (green) and B (blue) will be described.

As shown in FIG. 2 and FIG. 3, in the imaging device 1 with this structure, light that is incident from the side of the third secondary light detection pixel 30 (the opposite side of the imaging device 1 from the side at which the substrate 2 is disposed) reaches the photoelectric conversion portion 34 of the third secondary light detection pixel 30, and of the incident light, red light is selectively absorbed. Positive-negative charge, which is to say electron-hole pairs, is generated in accordance with the intensity of this red light. A predetermined voltage is applied between the lower electrode (the pixel electrode) and the upper electrode, the electrons are moved towards, for example, the lower electrode by the electric field that is generated in the photoelectric conversion portion, and these electrons are accumulated at the lower electrode. When the TFT provided in the third secondary light detection pixel 30 is turned on, the electrons accumulated at the lower electrode are outputted as a red light signal charge.

Hence, light that has not been absorbed by the photoelectric conversion portion 34 of the third secondary light detection pixel 30, which is to say light outside the red light wavelength region, is incident on the second secondary light detection pixel 20. At the second secondary light detection pixel 20, light in the green wavelength region is absorbed at the photoelectric conversion portion (light detection element) 24. The absorbed green light is photoelectrically converted by a similar action to that of the third secondary light detection pixel 30 for red light, and when the TFT provided in the second secondary light detection pixel 20 is turned on, is outputted as a green light signal charge.

Hence, light that has not been absorbed in the third and second secondary light detection pixels 30 and 20, which is to say blue light, is incident on the first secondary light detection pixel 10. At the first secondary light detection pixel 10, light in the blue wavelength region is absorbed at the photoelectric conversion portion (light detection element) 14. The absorbed blue light is photoelectrically converted by a similar action to those of the third and second secondary light detection pixels 30 and 20 for red light and green light, and when the TFT provided in the first secondary light detection pixel 10 is turned on, is outputted as a blue light signal charge.

Thus, because the secondary light detection pixels (light-receiving pixels) 10, 20 and 30, which sense and photoelectrically convert lights of respectively different wavelength regions, are layered on the substrate 2 in states which are insulated by the sealing insulation films 18 and 28 being interposed and the primary light detection pixels 4 structured by the layered secondary light detection pixels 10, 20 and 30 are arrayed on the substrate 2, signal charges corresponding to the respective wavelength regions (R, G, B) can be respectively outputted. Hence, by combining the signals outputted from the secondary light detection pixels 10, 20 and 30, a subject (the interior of a living body) can be imaged in full color with a high resolution. Moreover, because the sealing insulation films 18 and 28 between the secondary light detection pixels 10, 20 and 30 that are neighboring in the thickness direction have thicknesses which are much thinner than the substrate 2 supporting the whole of the light detection pixels, image fuzziness which would tend to occur if substrates (intermediate substrates) were disposed between the secondary light detection pixels 10, 20 and 30 can be effectively suppressed.

Moreover, because the semiconductor layer 48 of the field effect thin film transistor 40 which drives each of the secondary light detection pixels 10, 20 and 30 is formed of an oxide semiconductor or an organic semiconductor, light permeation higher than with a semiconductor layer formed of, for example, amorphous silicon, and larger currents can flow at low voltages. Therefore, detected light amounts at the respective secondary light detection pixels 10, 20 and 30 are improved, and imaging with high sensitivity is possible, in addition to which power consumption can be reduced. Moreover, the semiconductor layer 48 formed of an oxide semiconductor or an organic semiconductor can be formed by, for example, sputtering in the case of an oxide semiconductor, and by, for example, a vacuum deposition method in the case of an organic semiconductor. Therefore, the semiconductor layer 48 can be formed at respective low temperatures. Consequently, as well as high-endurance substrates of glass or the like, plastic substrates with flexibility may be excellently utilized as the substrate 2. Accordingly, this can contribute to a reduction in size and a reduction in weight of the capsule endoscope 100 into which this imaging device 1 is incorporated.

Next, operation of the present exemplary embodiment will be described.

As shown in FIG. 2, in the imaging device 1 of the capsule endoscope 100, the signal output section 12 and the photoelectric conversion portion 14 are superposed in plan view. Therefore, in comparison with, for example, a structure in which a signal output portion and a photoelectric conversion portion are not superposed (see, for example, FIG. 2 in JP-A No. 2007-105236), a projected area of the secondary light detection pixels 10, 20 and 30 in plan view can be reduced.

Further, because the secondary light detection pixels 10, 20 and 30 are layered on the substrate 2 with the sealing insulation films 18 and 28 interposed, a projected area of the imaging device 1 in plan view is not widened even for color imaging. Therefore, comparison with an imaging device with a structure in which, for example, the secondary light detection pixels 10, 20 and 30 (i.e., the primary light detection pixel 4) are laid out in in-plane directions, size is reduced.

As the respective secondary light detection pixels 10, 20 and 30 are reduced in size, and are layered, thus the imaging device 1 is reduced in size. As a result, the capsule endoscope 100 capable of capturing color images, which is equipped with the imaging device 1, is reduced in size.

Because the secondary light detection pixels 10, 20 and 30 are reduced in size and layered, the primary light detection pixels 4 can be provided at high density in the imaging device 1 (see FIG. 1). Therefore, an increase in resolution is possible even with the imaging device 1, that is, the capsule endoscope 100, being reduced in size (or not increased in size).

As shown in FIG. 5 and FIG. 6, at the field effect thin film transistor 40, the semiconductor layer 48 includes at least the resistance layer 48a and the active layer 48b with a greater electrical conductivity than the resistance layer 48a, the active layer 48b is in contact with the gate insulation film 46, and the resistance layer 48a is structured to electronically connect the active layer 48b with at least one of the source electrode 50 and the drain electrode 52.

Therefore, in an ON state of the field effect thin film transistor 40, in which a voltage is applied to the source electrode 50, because the active layer 48b has a large electrical conductivity, an electric field mobility is high, and a high ON current is provided. In an OFF state, because the electrical conductivity of the resistance layer 48a is small and the resistance of the resistance layer 48a is high, an OFF current is kept small. Therefore, an ON/OFF comparison characteristic is extremely good. As a result, imaging is performed with high resolution and high sensitivity. Moreover, power consumption is reduced.

Accordingly, because the power consumption is low, the capsule endoscope 100 of the present exemplary embodiment can perform imaging over a long period without carrying a large battery 164 (see FIG. 8). Thus, a further reduction in size is enabled.

Since the capsule endoscope 100 of the present exemplary embodiment is reduced in size, an imposition on the patient when swallowing the capsule endoscope 100 through the mouth and impositions while the capsule endoscope 100 is within the body are reduced. Furthermore, because resolution and sensitivity are improved, the interior of the body can be imaged with higher quality.

Furthermore, the signal output section 12 does not require a light-blocking layer for blocking light in the present exemplary embodiment, as it would in a case in which, for example, a CMOS sensor was used as the imaging device. Thus, there are effects of a reduction in noise due to leakage light and dark current. Moreover, a simplification of fabrication steps of the capsule endoscope 100 is enabled.

The present invention is not limited to the exemplary embodiment described above.

In the exemplary embodiment described above, as shown in FIG. 3, the region of the photoelectric conversion portion 14 is superposed so as to be accommodated by the signal output section 12 in plan view, but this is not a limitation. It is sufficient for at least a portion of the photoelectric conversion layer 15 and the signal output section 12 to be superposed.

In the exemplary embodiment described above, as shown in FIG. 8, the capsule endoscope 100 is equipped with transmission unit (i.e., the transmission/reception section 168). However, in the case of a structure in which, for example, image data can be saved within the capsule, a structure in which the transmission means is not provided is also possible.

The exemplary embodiment described above has a structure in which, for example, the secondary light detection pixels 10, 20 and 30 are layered for imaging the interior of a body in color. However, this is not a limitation. For example, the primary light detection pixels may be structured by layering two kinds of secondary light detection pixels, and the primary light detection pixels may be structured by layering four or more kinds of secondary light detection pixels. Alternatively, a structure with single secondary light detection pixels is possible.

In the exemplary embodiment described above, as shown in FIG. 7A, the capsule endoscope 100 is swallowed by a patient K and is used for the purpose of imaging the interior of the body of the patient K. However, imaging objects (subjects) are not limited thus. The capsule endoscope 100 may be used for purposes of imaging various subjects other than the interior of a living body. Application to industrial purposes such as, for example, imaging interior walls of pipes and the like is possible.

In the exemplary embodiment described above, as shown in FIG. 8, the capsule endoscope 100 is equipped with illumination means (the white LEDs 158A and 158B). However, in the case of a structure in which, for example, a subject (target location) will have a brightness that can be imaged without being illuminated, illumination means will be provided separately from the capsule endoscope, or the like, a structure that is not equipped with illumination means is possible.

Next, details of members constituting the imaging device 1, and details of fabrication processes of the imaging device 1 will be described.

—Substrate—

A material of the substrate 2 is not particularly limited and, for example, the following can be used: YSZ (yttria-stabilized zirconia); inorganic materials such as glass and the like; organic materials of synthetic resins such as polyesters including polyethylene terephthalate, polybutylene terephthalate, polyethylene naphthalate and the like, polystyrene, polycarbonate, polyether sulfone, polyarylate, aryl diglycol carbonate, polyimide, polycycloolefin, norbornene resin, poly(chlorotrifluoroethylene) and the like. Cases of organic materials are preferable in view of excellence of light permeation, heat endurance, dimensional stability, surface flatness, solvent resistance, electrical insulation, machining characteristics, air permeability, moisture absorption and so forth.

For the imaging device 1 of the present exemplary embodiment, in particular the substrate 2, a flexible substrate (bendable substrate) may be preferably used. As a material used for the substrate 2, a plastic film with high light permeation is preferable, and a plastic-form film of the above-mentioned organic materials may be excellently used. Further, for a substrate 2 employing a film-form plastic, it is preferable to provide: an insulation layer if insulation would be insufficient; a gas barrier layer for preventing permeation of water, oxygen and the like; an undercoat layer for improving flatness of the substrate 2 and adherence of the field effect thin film transistor 40; and so forth.

In a case in which a flexible substrate is employed, while a thickness thereof will depend on the material, a thickness with which it is both possible to reliably support the light detection pixels formed on the substrate 2 and possible to freely bend the substrate 2 is preferable, and may be, for example, from 10 μm to 1 mm, more preferably from 20 μm to 0.5 mm.

When such a flexible substrate 2 made of plastic is employed, it can be freely deformed by bending, curling and the like, which enables a contribution to a reduction in size and a reduction in weight of the device.

As shown in FIG. 2, in a case in which light is received to be photoelectrically converted from the third secondary light detection pixel 30 side, which is the opposite side from the substrate 2, there is no need for the substrate 2 to be transparent, and a non-transparent substrate may be employed, such as, for example, a metal substrate, a semiconductor substrate or the like.

On the other hand, in a case in which light is received and the secondary light detection pixels 10, 20 and 30 sense the light from the opposite side to that in FIG. 2 (i.e., that in the present exemplary embodiment), that is, from the side at which the substrate 2 is disposed, a substrate 2 with high light permeation will be employed. In such a case, although it will depend on required sensitivity and the like, the substrate 2 will preferably have as high a light permeation as possible.

—Field Effect Thin Film Transistor—

As has already been described, the first secondary light detection pixel 10 shown in FIG. 3 outputs a signal from the signal output section 12 including the capacitor 60 and the field effect thin film transistor 40 on the basis of a charge produced by the photoelectric conversion portion 14. The field effect thin film transistor 40 includes the gate electrode 44, the gate insulation film 46, the semiconductor layer 48, the source electrode 50 and the drain electrode 52, and the semiconductor layer 48 is formed of an oxide semiconductor or an organic semiconductor. The second and third secondary light detection pixels 20 and 30 are provided with the signal output sections 22 and 32 that include field effect thin film transistors with respectively similar structures, and output signals on the basis of charges produced by the respective photoelectric conversion portions 24 and 34 (see FIG. 2).

—Semiconductor Layer—

If the semiconductor layer 48 is formed of an oxide semiconductor, charge mobility will be much higher than in a semiconductor layer of amorphous silicon, and can be driven by low voltages. Further, when an oxide semiconductor is used, light permeation will usually be higher than with silicon, and the semiconductor layer 48 can be formed to have flexibility. With an oxide semiconductor, particularly an amorphous oxide semiconductor, uniform film formation at a low temperature (for example, room temperature) is possible. Therefore, this is particularly advantageous when using a substrate 2 made of a resin that is flexible such as a plastic. Because the plural secondary light detection pixels are layered, a lower level secondary light detection pixel would be affected when an upper level secondary light detection pixel is formed. In particular, a photoelectric conversion layer is easily influenced by heat, but an oxide semiconductor, particularly an amorphous oxide semiconductor, can form a film at low temperature therefore, this is advantageous to reduce the heat influence to the photoelectric conversion layer.

As an oxide semiconductor for forming the semiconductor layer 48, an oxide including at least one of In, Ga and Zn (for example, an In—O type) is preferable, an oxide including at least two of In, Ga and Zn (for example, an In—Zn—O type, an In—Ga—O type or a Ga—Zn—O type) is more preferable, and an oxide including In, Ga and Zn is even more preferable. As an In—Ga—Zn—O type Oxide semiconductor, an oxide semiconductor of which the composition in a crystalline state is represented by InGaO₃(ZnO)ₘ (m is a natural number less than 6) is preferable, and in particular, InGaZnO₄ is more preferable. The characteristics of amorphous oxide semiconductors of such compositions exhibit electrical mobility to increase as electrical conductivity increases.

Note that electrical conductivity is a physical value representing electrical conductivity in a material. If a carrier density in the material is n and a carrier mobility is μ, an electrical conductivity a of the material is shown by the following equation, in which e represents the elementary charge.

σ=neμ

If the semiconductor layer 48 is an n-type semiconductor, the carriers are electrons, the carrier density represents an electron carrier density, and the carrier mobility represents electron mobility. Similarly, if the semiconductor layer 48 is a p-type semiconductor, the carriers are holes, the carrier density represents a hole carrier density, and the carrier mobility represents hole mobility. The carrier density and carrier mobility of a material can be found by Hall's Law.

For the electrical conductivity, the electrical conductivity of a film whose thickness is known can be obtained by measuring a sheet resistance of the film. The electrical conductivity of a semiconductor varies with temperature, but in the present embodiments, electrical conductivity refers to electrical conductivity at room temperature (20° C.).

As an oxide semiconductor forming the semiconductor layer 48, as mentioned above, an oxide including at least one of In, Ga and Zn is preferable, and a p-type semiconductor such as ZnO.Rh2O₃, CuGaO₂ or SrCuO₂ may be used for the semiconductor layer 48.

The electrical conductivity of the semiconductor layer 48 is preferably higher in a vicinity of the gate insulation film 46 than in vicinities of the source electrode 50 and the drain electrode 52. More preferably, a ratio of electrical conductivity in the vicinity of the gate insulation film 46 to electrical conductivity in the vicinities of the source electrode 50 and the drain electrode 52 (i.e., electrical conductivity in the vicinity of the gate insulation film 46/electrical conductivity in the vicinities of the source electrode 50 and the drain electrode 52) is preferably from 10¹ to 10¹⁰, and is more preferably from 10² to 10⁸. It is preferable if electrical conductivity in the vicinity of an electrical field at the gate insulation film 46 of the semiconductor layer 48 is from 10⁻⁴ S·cm⁻¹ to 10² S·cm⁻¹, and this is more preferably 10⁻¹ S·cm⁻¹ to 10² S·cm⁻¹.

The semiconductor layer 48 may be formed in plural layers. For example, as shown in FIG. 5, it is preferable that the semiconductor layer 48 includes at least the resistance layer 48a and the active layer 48b with a greater electrical conductivity than the resistance layer 48a, that the active layer 48b is in contact with the gate insulation film 46, and that the resistance layer 48a is structured to electronically connect the active layer 48b with at least one of the source electrode 50 and the drain electrode 52. More preferably, a ratio of electrical conductivity of the active layer 48b to electrical conductivity of the resistance layer 48a (i.e., electrical conductivity of the active layer 48b/electrical conductivity of the resistance layer 48a) is from 10¹ to 10¹⁰, and is even more preferably from 10² to 10⁸.

Preferably, the electrical conductivity of the active layer 48b is from 10⁻⁴ S·cm⁻¹ to 10² S·cm⁻¹, and this is more preferably 10⁻¹ S·cm⁻¹ to 10² S·cm⁻¹. The electrical conductivity of the resistance layer 48a is preferably less than 10⁻² S·cm⁻¹, and more preferably 10⁻⁹ S·cm⁻¹ to 10⁻³ S·cm⁻¹.

A film thickness of the resistance layer is preferably thicker than a film thickness of the active layer. More preferably, a resistance layer film thickness/active layer film thickness ratio is greater than 1 and less than 100, and more preferably greater than 1 and less than 10.

The film thickness of the active layer is preferably from 1 nm to 100 nm, and more preferably from 2.5 nm to 30 nm. The film thickness of the resistance layer is preferably from 5 nm to 500 nm, and more preferably from 10 nm to 100 nm.

If a two-layer structure of the resistance layer 48a and the active layer 48b is formed of an amorphous oxide semiconductor such as IGZO or the like as mentioned above, a high-mobility TFT with a mobility of 10 cm²/(V·s) or greater and a transistor characteristic with an ON/OFF ratio of 10⁶ or more can be realized, and a further reduction in voltages can be achieved.

The field effect thin film transistor provided at each of the secondary light detection pixels 10, 20 and 30 may be either of a bottom-gate type and a top-gate type. For example, as shown in FIG. 6, a field effect thin film transistor that is structured with the source and drain electrodes 50 and 52, the active layer 48b, the resistance layer 48a, the gate insulation film 46 and the gate electrode 44 being layered in this order from the substrate 2 side may be formed.

In FIG. 5 and FIG. 6, an insulation film 3 is formed on the substrate 2, and the field effect thin film transistor is formed thereon. Particularly in a case in which a substrate with conductivity is employed, such as a metal substrate or a semiconductor substrate or the like, an insulation layer may be formed thus and an insulated substrate provided.

As mentioned above, the semiconductor layer 48 relating to the present exemplary embodiment is preferably arranged such that electrical conductivity is greater in the vicinity of the gate insulation film 46 than in the vicinities of the source electrode 50 and the drain electrode 52 of the semiconductor layer 48. A mode in which, for example, electrical conductivity varies continuously between the resistance layer and the active layer is also preferable. There is not distinct border between the resistance layer and the active layer in this structure. A region of 10% of a total thickness of a semiconductor layer, which combines the resistance layer and the active layer, that is closer to the gate insulation film will be defined as the active layer, and a region of 10% of the thickness of this semiconductor layer that is closer to the source electrode and drain electrode will be defined as the resistance layer.

For cases in which the semiconductor layer 48 is formed with an oxide semiconductor, the following techniques can be used for adjusting electrical conductivity.

(1) Adjustment by Oxygen Deficit

It is known that when there is an oxygen deficit in an oxide semiconductor, carrier electrons are generated, and electrical conductivity is greater. Therefore, the electrical conductivity of an oxide semiconductor can be controlled by adjusting an oxygen deficit amount. As specific methods for controlling the oxygen deficit amount, conducting oxygen partial pressure during film formation and arranging oxygen concentration or processing duration and the like during post-processing after film formation can be employed. Post-processing here specifically means heating processing at over 100° C., oxygen plasma, UV ozone processing and the like. Among these methods, the method of controlling oxygen partial pressure during film formation is preferable in regard to productivity. Thus, control of electrical conductivity of the oxide semiconductor may be implemented by adjusting an oxygen partial pressure during film formation.

(2) Adjustment by Composition Ratio

Electrical conductivity can varied by changing a metal composition ratio in an oxide semiconductor. For example, with InGaZn₁₋ₓMgₓO₄, if the proportion of Mg is increased, the electrical conductivity decreases. Further, it has been reported that in an oxide such as (In₂O₃)₁₋ₓ(ZnO)ₓ, when the Zn/In ratio is above 10%, the electrical conductivity decreases as the proportion of Zn increases (“New Developments in Transparent Conductive Films II”, CMC publishing, pp 34-35). Preferably, the Zn/In ratio in the resistance layer is at least 3% larger than the Zn/In ratio in the active layer, and is more preferably at least 10% greater.

As a specific method for varying such a composition ratio in a process of film formation by sputtering, for example, a method of using targets with different composition ratios can be employed. By sputtering with plural targets and separately adjusting sputter rates, it is also possible to vary composition ratios in a film.

(3) Adjustment by Impurities

By adding an element such as Li, Na, Mn, Ni, Pd, Cu, Cd, C, N, P or the like as an impurity in an oxide semiconductor, the electron carrier density can be reduced; that is, electrical conductivity can be reduced. As methods for adding impurities, there are implementations by co-sputtering of the oxide semiconductor and an impurity element, by ion-doping ions of the impurity element into the oxide semiconductor which has been formed into a film, and suchlike.

(4) Adjustment by Oxide Semiconductor Materials

In (1) to (3) above, methods of adjusting electrical conductivity in the same kind of oxide semiconductor have been described, but it is of course possible to vary electrical conductivity by changing the oxide semiconductor material. For example, it is known that a Sn0₂ oxide semiconductor has a smaller electrical conductivity than an In₂0₃ oxide semiconductor. By changing the oxide semiconductor in such a manner, adjustment of electrical conductivity is possible. In particular, as oxide semiconductors with small electrical conductivities, oxide insulator materials such as Al₂O₃, Ga₂O₃, ZrO₂, Y₂O₃, Ta₂O₃, MgO, HfO₃ and the like are known, and these may be employed.

As a technique for adjusting electrical conductivity, the above-described methods (1) to (4) may be employed singly, and may be combined.

As a method for forming the semiconductor layer 48, a vapor phase film formation method using a polycrystalline sintered body of the oxide semiconductor as a target may be used. Of vapor phase film formation methods, a sputtering method and a pulse laser vapor deposition method (PLD method) are applicable. With regard to productivity, the sputtering method is preferable.

In, for example, an RF magnetron sputtering vapor deposition process, a film is formed with a degree of vacuum and an oxygen flow amount being controlled. The greater the oxygen flow amount, the smaller the electrical conductivity that results.

As for adjusting electrical conductivity during film formation, the above-described methods (1) to (4) may be employed singly, and may be combined.

A film which has been formed can be confirmed to be an amorphous film with, for example, a widely known X-ray diffraction technique.

A film thickness can be found by stylus profile measurement, and a composition ratio can be found with an RBS (Rutherford back-scattering) analysis technique.

The semiconductor layer 48 may also be formed of an organic semiconductor. Organic semiconductors such as various condensed polycyclic aromatic compounds, conjugated compounds and the like which can form films at low temperature and have conductivity and light permeation may be employed.

Specifically, as a low polymer semiconductor, the following may be employed: acene compounds as represented by pentacene, tetracene and anthracene; phthalocyanine pigments as represented by non-metallic Or bivalent phthalocyanines with Cu, Zn, Co, Ni, Pb, Pt, Fe, Mg or the like as a core metal, trivalent metallic phthalocyanines coordinated with halogen atoms such as aluminum chlorophthalocyanine, indium chlorophthalocyanine, gallium chlorophthalocyanine and the like, and also phthalocyanines coordinated with oxygen such as vanadyl phthalocyanine, titanyl phthalocyanine and the like; indigo and thioindigo pigments; quinacridone pigments; perylene pigments such as perylene, PTCDA, PTCDI, PTCBI, Me-PTC and the like; C60, C70, C76, C78, C84 and other fullerenes; carbon nanotubes; color pigments such as melocyanine color pigments and the like; and the like.

As a high polymer semiconductor, the following may be employed: polypyrroles such as polypyrrole, poly(N-substituted pyrrole) and the like; polythiophenes such as polythiophene, poly(3-substituted thiophene) and the like; polyacetylenes; and polymers such as polyvinyl carbazole, polyphenylene sulfide, polyvinylene sulfide and the like.

The above-mentioned materials may be used singly, and may be employed by being dispersedly mixed in a binder such as a resin or the like and then used.

In order to adjust conductivity of an organic semiconductor, it may be doped with a dopant such as a donor-type or acceptor-type non-organic material, non-organic compound, organic compound or the like.

As a method for forming the semiconductor layer 48 of an organic semiconductor, a dry film formation method or a wet film formation method may be employed. As specific examples of dry film formation methods, a physical vapor phase growth method such as a vacuum deposition method, a sputtering method, an ion plating method, an MBE method or the like, and a CVD method such as a plasma polymerization method or the like can be employed. As a wet film formation method, a coating method such as a casting method, a spin-coating method, a dipping method, an LB method or the like may be used. Furthermore, printing methods such as inkjet printing, screen printing or the like, and transcription methods such as thermal transcription, laser transcription or the like may be used. Patterning may be implemented by: chemical etching by photolithography or the like; physical etching with ultraviolet radiation, a laser or the like; performing vapor deposition, sputtering or the like with a mask superposed; a lift-off method; a printing method; or a transcription method.

In a case in which a low polymer organic semiconductor is employed, a dry film formation method is preferably employed, and in particular, a vacuum deposition method is preferably employed. In a vacuum deposition method, the basic parameters are: a method of heating a compound such as a resistance heating deposition method, an electron beam heating deposition method or the like; the form of a deposition source such as a crucible, a board or the like; a degree of vacuum; a deposition temperature; a substrate temperature; a deposition rate; and the like. To enable uniform deposition, it is preferable to perform deposition while turning the substrate 2. A higher degree of vacuum is preferable, and the vacuum deposition is preferably performed under 10⁻⁴ Torr or less, preferably 10⁻⁶ Torr or less, and particularly preferably 10⁻⁸ Torr or less. It is preferable to carry out all steps at the time of deposition in a vacuum, so that the compound does not come into direct content with oxygen or moisture in the air. The above-mentioned conditions of the vacuum deposition affect crystallinity of the organic film, amorphousness, density, microdensity and the like, so must be strictly controlled. It is preferable to perform PI or PID control of the deposition rate by using a film thickness monitor such as a quartz oscillator, an interferometer or the like. In a case in which two or more kinds of compound are being deposited simultaneously, a co-deposition method, a flash deposition method or the like may be preferably used.

In a case of employing a high polymer semiconductor, film formation with a wet film formation method is preferable. A case of using a dry film formation method such as deposition or the like will be difficult because there is a risk of the polymer that is being used decomposing, but an oligomer thereof may be preferably used instead.

The thickness of the semiconductor layer 48 depends on the material that is used and the like, but is preferably from 10 nm to 1 μm, more preferably from 20 nm to 500 nm, and particularly preferably from 30 nm to 200 nm.

—Gate Insulation Layer—

For the gate insulation film 46, an inorganic compound, organic compound or the like with a high relative permittivity may be employed.

As an inorganic compound, the following may be employed: silicon oxide, silicon nitride, germanium oxide, germanium nitride, aluminum oxide, aluminum nitride, yttrium oxide, tantalum oxide, hafnium oxide, silicon oxynitride, silicon oxycarbide, silicon nitrocarbide, silicon oxynitrocarbide, germanium oxynitride, germanium oxycarbide, germanium nitrocarbide, germanium oxynitrocarbide, aluminum oxynitride, aluminum oxycarbide, aluminum nitrocarbide, aluminum oxynitrocarbide, and mixtures thereof.

As an organic compound, a polyimide, a polyamide, a polyester, a polyacrylate, a copolymer including a light radical-polymerizable or light cation-polymerizable light-curable resin or an acrylonitrile component, a polyvinyl phenol, polyvinyl alcohol, novolac resin, cyanoethyl pullulan or the like may be employed. Further, a powder in which microparticles of such a polymer are covered with an inorganic oxide may be employed.

As a method for forming the gate insulation film 46, a dry film formation method or a wet film formation method may be employed. As specific examples of dry film formation methods, a physical vapor phase growth method such as a vacuum deposition method, a sputtering method, an ion plating method, an MBE method or the like, and a CVD method such as a plasma polymerization method or the like can be mentioned. As a wet film formation method, a coating method such as a casting method, a spin-coating method, a dipping method, an LB method or the like may be used. Furthermore, printing methods such as inkjet printing, screen printing or the like, and transcription methods such as thermal transcription, laser transcription or the like may be used. Patterning may be implemented by: chemical etching by photolithography or the like; physical etching with ultraviolet radiation, a laser or the like; a vacuum deposition, sputtering or the like with a mask superposed; a lift-off method; a printing method; or a transcription method.

Although it depends on the configuration of the TFT 40, the gate insulation film 46 may be formed by a method of oxidizing a surface of the gate electrode 44 by O₂ plasma processing, an anode oxidation method or the like, or a method of nitriding using N₂ plasma, or the like.

A film thickness of the gate insulation film 46 is preferably 30 nm to 3 μm, and more preferably 50 nm to 1 μm.

—Gate Electrode, Source Electrode and Drain Electrode—

The gate electrode 44, the source electrode 50 and the drain electrode 52 are not particularly limited as long as they are conductive materials. For example, the following can be employed: platinum, gold, silver, nickel, chromium, copper, iron, tin, antimony, tantalum, indium, aluminum, zinc, magnesium, alloys of these metals, conductive metal oxides such as indium tin oxide (ITO), indium zinc oxide (IZO) and the like, inorganic and organic semiconductors whose conductivity has been improved by doping or the like (silicon monocrystal, polysilicon, amorphous silicon, germanium, graphite, polyacetylene, polyparaphenylene, polythiophene, polypyrrole, polyaniline, polyphenylene vinylene, polyparaphenylene vinylene and the like), and complexes of these materials. In particular, of the above materials, electrode materials used for the source electrode and the drain electrode will preferably have low electrical resistance at least at a contact surface with the semiconductor layer 48.

Particularly in a case in which a flexible substrate made of plastic is used, it will be preferable to form the respective electrodes 44, 50 and 52 using a material capable of film formation at a low temperature, for example, a conductive metal oxide such as indium tin oxide (ITO), indium zinc oxide (IZO) or the like, or an organic semiconductor whose conductivity has been improved by doping or the like. If such materials are used, it will be possible to form the whole of the field effect thin film transistor 40 by low temperature processes, and it will be possible to form the field effect thin film transistor 40 with higher light permeation and flexibility. Herein, it is preferable for the field effect thin film transistor 40 to have higher light permeation. Specifically, it is preferable for a permeation of visible light to be at least 60%, more preferably at least 70%, and particularly preferably at least 80%. The higher the light permeation of the field effect thin film transistor 40 at each of the secondary light detection pixels 10, 20 and 30, the better light detection amounts at the photoelectric conversion layer 15 will be, and the higher the sensitivity.

Furthermore, if the electrodes 13 and 16 of the photoelectric conversion portion (light detection element) 14 are formed of materials capable of film formation at low temperatures as described above, the whole of the secondary light detection pixels may be reliably formed by low temperature processes, which is particularly advantageous when employing a flexible substrate 2.

As a method for forming the gate electrode 44, a dry film formation method or a wet film formation method may be employed. As specific examples of dry film formation methods, a physical vapor phase growth method such as a vacuum deposition method, a sputtering method, an ion plating method, an MBE method or the like, and a CVD method such as a plasma polymerization method or the like can be mentioned. As a wet film formation method, a coating method such as a casting method, a spin-coating method, a dipping method, an LB method or the like may be used. Furthermore, printing methods such as inkjet printing, screen printing or the like, and transcription methods such as thermal transcription, laser transcription or the like may be used.

Patterning may be implemented by: chemical etching by photolithography or the like; physical etching with ultraviolet radiation, a laser or the like; performing vapor deposition, sputtering or the like with a mask superposed; a lift-off method; a printing method; or a transcription method. From these film formation methods and patterning methods, selections may be made in consideration of the material to be employed, the material of the substrate 2 and the like.

For formation of the source electrode 50 and the drain electrode 52, similar methods to the gate electrode 44 may be employed.

Respective film thicknesses of the gate electrode 44, the source electrode 50 and the drain electrode 52 are each preferably from 10 nm to 1 μm, more preferably from 30 nm to 500 nm, and particularly preferably from 50 nm to 200 nm.

—Capacitor—

The capacitor 60 shown in FIG. 3 is electrically connected with the corresponding lower electrode (pixel electrode) 13 by wiring of a conductive material, which is formed passing through an insulation film 54 that is provided between the substrate 2 and the lower electrode 13. Thus, charge trapped in the lower electrode 13 can be moved to the capacitor 60.

The capacitor 60 is structured by an insulated pair of electrodes 64 and 66, and may be formed by patterning with photolithography or the like at the same times as when the gate electrode 44, the gate insulation film 46 and the source and drain electrodes 50 and 52 of the field effect thin film transistor 40 are being formed. Here, the upper electrode 66 of the capacitor 60 is patterned so as to be electrically connected with the drain electrode 52. The upper electrode 66 of the capacitor 60 is also electrically connected with the lower electrode 13 of the photoelectric conversion portion 14 by a through-hole 70.

—Interlayer Insulation Film—

After the field effect thin film transistor 40 and the capacitor 60 have been formed, the protective film (interlayer insulation film) 54 is formed. An inorganic compound or organic compound the same as the gate insulation film 46 may be used for the interlayer insulation film 54.

As a method for forming the interlayer insulation film 54, a dry film formation method or a wet film formation method may be employed. As specific examples of dry film formation methods, a physical vapor phase growth method such as a vacuum deposition method, a sputtering method, an ion plating method, an MBE method or the like, and a CVD method such as a plasma polymerization method or the like can be mentioned. As a wet film formation method, a coating method such as a casting method, a spin-coating method, a dipping method, an LB method or the like may be used. Furthermore, printing methods such as inkjet printing, screen printing or the like, and transcription methods such as thermal transcription, laser transcription or the like may be used. Patterning may be implemented by: chemical etching by photolithography or the like; chemical etching with ultraviolet radiation, a laser or the like; performing vapor deposition, sputtering or the like with a mask superposed, and may be implemented by a lift-off method, a printing method or a transcription method.

The interlayer insulation film 54 (protective film), in which a contact hole is provided, may be formed by, for example, coating an acryl-based photoresin onto the substrate 2 using a spin-coater or the like, exposing such that the contact hole is formed at a predetermined position, and then developing.

A film thickness of the interlayer insulation film 54 is preferably 50 nm to 3 μm, and more preferably 10 nm to 1 μm.

—Lower Electrode and Upper Electrode—

Of the lower electrode (pixel electrode) 13 and upper electrode (counter electrode) 16 which constitute the photoelectric conversion portion (light detection element), one serves as an anode and the other serves as a cathode.

Lower electrodes and upper electrodes 16, 26 and 36 of the respective secondary light detection pixels 10, 20 and 30 need to be transparent or semi-transparent, preferably having light transparencies for the wavelength region of visible light, which is from 400 nm to 700 nm, of at least 50%, preferably at least 70%, and more preferably at least 90%.

Materials of these electrodes are selected in consideration of, as well as light permeation and conductivity, adherence and electronic affinity with neighboring layers, ionic potential, stability and the like. Metals, alloys, metal oxides, electrically conductive compounds, mixed materials thereof, and the like may be employed.

More specifically, the following can be mentioned: conductive metal oxides such as tin oxide, zinc oxide, indium oxide, indium tin oxide (ITO), IZO, AZO, FTO, SnO₂, TiO₂, ZnO₂ and the like; metals such as gold, silver, chromium, nickel and the like; mixtures and layers of these metals and metal oxides; inorganic conductive materials such as copper iodide, copper sulfide and the like; organic conductive materials such as polyaniline, polythiophene, polypyrrole and the like; silicon compounds and layers thereof with ITO; or the like. As a conductive material requiring high light permeation, conductive metal oxides are preferable, and in particular, with regard to productivity, conductivity, permeation and the like, ITO and IZO are preferable.

As a method for forming the lower electrode (pixel electrode) 13 and upper electrode (counter electrode) 16, a dry film formation method or a wet film formation method may be employed. As specific examples of dry film formation methods, a physical vapor phase growth method such as a vacuum deposition method, a sputtering method, an ion plating method, an MBE method or the like, and a CVD method such as a plasma polymerization method or the like can be mentioned. As a wet film formation method, a coating method such as a casting method, a spin-coating method, a dipping method, an LB method or the like may be used. Furthermore, printing methods such as inkjet printing, screen printing or the like, and transcription methods such as thermal transcription, laser transcription or the like may be used. Patterning may be implemented by: chemical etching by photolithography or the like; physical etching with ultraviolet radiation, a laser or the like; performing vapor deposition, sputtering or the like with a mask superposed; a lift-off method; a printing method; or a transcription method.

For the lower electrode (pixel electrode) 13, after film formation, the lower electrode 13 may be formed while being set apart in each primary light detection pixel by performing patterning. The upper electrode (counter electrode) 16 may be formed as a single-sheet structure for all the pixels, or may be set apart for the respective primary light detection pixels.

Film thicknesses of the electrodes 13 and 16 may be suitably selected in accordance with the materials, but should be as thin as possible in order to raise light permeation. Ordinarily, a range from 3 nm to 500 nm is preferable, 5 nm to 300 nm is more preferable, and 7 nm to 100 nm is even more preferable. Sheet resistances of the anode and cathode are preferably lower, preferably being lower than a matter of hundreds of Ω/square.

—Photoelectric Conversion Layer—

The photoelectric conversion layer 15 of the photoelectric conversion portion 14 is structured such that the layered three kinds of secondary light detection pixels 10, 20 and 30 sense and photoelectrically convert light of respectively different wavelength regions.

The photoelectric conversion layer of each of the secondary light detection pixels 10, 20 and 30 may employ a photoelectric conversion material that absorbs light of a respective predetermined wavelength region and generates charge in accordance with intensity of the light. Specifically, examples of organic materials that absorb and photoelectrically convert blue light include porphyrin derivatives, examples of organic materials that absorb and photoelectrically convert green light include perylene derivatives, and examples of organic materials that absorb and photoelectrically convert red light include phthalocyanine derivatives.

An organic material structuring a photoelectric conversion layer is not particularly limited to those mentioned above. For example, the photoelectric conversion layer 15 may be formed with one of the following: acridine, coumarin, quinacridone, cyanine, squarylium, oxazine, xanthene triphenylamine, benzidine, pyrazoline, styrylamine, hydrazone, triphenyl methane, carbazole, polysilane, thiophene, polyamine, oxadiazole, triazole, triazine, quinoxaline, phenanthroline, fullerene, aluminum quinoline, polyparaphenylene vinylene, polyfluorene, polyvinyl carbazole, polythiol, polypyrrole, polythiophene, derivatives thereof and the like. Alternatively, two or more of these representative materials may be mixed or layered.

As a method for forming the photoelectric conversion layer 15, a dry film formation method or a wet film formation method may be employed. As specific examples of dry film formation methods, a physical vapor phase growth method such as a vacuum deposition method, a sputtering method, an ion plating method, an MBE method or the like, and a CVD method such as a plasma polymerization method or the like can be mentioned. As a wet film formation method, a coating method such as a casting method, a spin-coating method, a dipping method, an LB method or the like may be used. Furthermore, printing methods such as inkjet printing, screen printing or the like, and transcription methods such as thermal transcription, laser transcription or the like may be used. Patterning may be implemented by: chemical etching by photolithography or the like; physical etching with ultraviolet radiation, a laser or the like; performing vapor deposition, sputtering or the like with a mask superposed; a lift-off method; a printing method; or a transcription method.

In order to reduce the dark current (a current that is observed when no light is being illuminated) and improve quantum efficiency, an electron transport material, a hole transport material, an electron blocking material and a hole blocking material or the like may be mixed in or layered. Such a layer may be formed by the same method as the photoelectric conversion layer 15.

Note that, cases of structuring such that three primary colors are detected by the layered secondary light detection pixels 10, 20 and 30 are not limited to the order blue light-green light-red light (BGR) from the side of the substrate 2 as described above. The three kinds of secondary light detection pixels 10, 20 and 30 may be formed so as to selectively sense light of wavelength regions corresponding to any of R, G and B respectively, and lights of the three primary colors photoelectrically converted in accordance with the combination thereof. Therefore, the photoelectric conversion layers 15 may be formed such that the three kinds of secondary light detection pixels 10, 20 and 30 can absorb and photoelectrically convert lights of the respective colors in any of these patterns, from the substrate 2 side: BGR, BRG, GBR, GRB, RGB and RBG.

—Sealing Insulation Film—

After the upper electrode 16 has been formed on the photoelectric conversion layer 15, the sealing insulation film 18 or 28 is formed. The sealing insulation film 18 or 28 is formed of a material having insulativity and light permeation. As a material for forming the sealing insulation film 18 or 28, for example, a material the same as that of the aforementioned gate insulation film 46 or interlayer insulation film 54 may be employed, and inorganic compounds are more preferable. As an inorganic compound for forming the sealing insulation film, for example, inorganic materials such as Al₂O₃, SiO₂, TiO₂, ZrO₂, MgO, HfO₂, Ta₂O₅, SiO (silicon oxide), SiON (silicon oxynitride), SiN (silicon nitride), AlN (aluminum nitride) and the like can be mentioned. It is preferable if the sealing insulation film 18 or 28 is an inorganic material formed by an atomic layer chemical vapor deposition method (ALCVD method).

Here, the sealing insulation film 18 interposed between the first and second secondary light detection pixels 10 and 20 is formed so as to permeate the respective lights (G and R) that will be sensed by the second and third secondary light detection pixels 20 and 30, and the sealing insulation film 28 interposed between the second and third secondary light detection pixels 20 and 30 is formed so as to permeate the light (R) that will be sensed by the third secondary light detection pixel 30.

As a method for forming the sealing insulation film 18 or 28, a dry film formation method or wet film formation method the same as in formation of the aforementioned gate insulation film 46 or interlayer insulation film 54 may be employed, and should be selected in consideration of the material to be used, the material of the substrate 2 and the like.

Thicknesses of the sealing insulation films 18 and 28 are preferably from 50 nm to 10 μm, are more preferably from 70 nm to 5 μm, and are most preferably from 100 nm to 3 μm. When such sealing insulation films 18 and 28 are provided between neighboring secondary light detection pixels, the respective secondary light detection pixels 10, 20 and 30 are kept in insulated states and can be independently controlled. The sealing insulation films 18 and 28 can be made much thinner than the substrate 2 that supports all the light detection pixels. Thus, the imaging device 1 does not employ intermediate substrates, and therefore gaps between the secondary light detection pixels 10, 20 and 30 are very small, and image fizziness can be effectively prevented.

—Smoothing Layer—

In addition to the sealing insulation films 18 and 28, it is preferable to layer secondary light detection pixels that are adjacent in the thickness direction with the smoothing layers 19 and 29, which are provided interposed on the sealing insulation films 18 and 28. Since patterning is performed by photolithography or the like when the field effect thin film transistor 40 of the first secondary light detection pixel 10 is being formed, surface irregularities reflecting this may arise at the surface of the sealing insulation film 18. If the field effect thin film transistor and the like of the second secondary light detection pixel 20 are formed on the sealing insulation film 18 at which such surface irregularities have arisen, formation problems, film thickening and the like may result. Therefore, after the sealing insulation film 18 has been formed on the first secondary light detection pixel 10, If the smoothing layer 19 is formed thereon and raises a degree of smoothness before the second secondary light detection pixel 20 is formed, formation problems and the like with the field effect thin film transistor and the like of the second secondary light detection pixel 20 can be effectively prevented. It is also preferable to similarly form the sealing insulation film 28 and smoothing layer 29 in this order after the second secondary light detection pixel 20 has been formed. Herein, there is no particular need to provide a smoothing layer after a sealing insulation film 38 has been formed on the third secondary light detection pixel 30 (the upper electrode 36).

The smoothing layer 19 or 29 is formed of a material with insulativity and light permeation. Specifically, a material the same as the gate insulation film 46 or the interlayer insulation film 54 may be employed, and an organic layer formed of an organic polymer is particularly preferable. An organic polymer is a high polymer film of fluoride resin, polyparaxylene, polyethylene, silicon resin, polystyrene resin or the like, and photocurable resins are more preferable.

As a method for forming the smoothing layer 19 or 29, various CVD methods can be mentioned: for example, a plasma-assisted method, an IPC-CVD method, a Cat-CVD method or an atomic layer CVD method (ALCVD method). With such a method, even if surface irregularities are formed at the sealing insulation film 18 or 28, a smoothing layer with a high degree of smoothness can be formed.

Thicknesses of the smoothing layers 19 and 29 are preferably from 50 nm to 10 μm, more preferably from 70 nm to 5 μm, and particularly preferably from 100 nm to 3 μm. With smoothing layers 19 and 29 of such thicknesses, degrees of smoothness can be improved, and reductions in light permeation and widening of gaps between the secondary light detection pixels can be effectively restrained.

Note that the sealing insulation films 18 and 28 and the smoothing layers 19 and 29 may employ the same materials, and the sealing insulation films 18 and 28 may be formed in combination with the smoothing layers 19 and 29. For example, if film formation is performed for a multilayer structure by a plasma CVD method using SiN (silicon nitride) and SiO (silicon oxide), the sealing insulation films 18 and 28 can be formed with both barrier characteristics and softness being provided, and with high microdensity, good permeation, and high degrees of smoothness.

By processes as described above, at the side face of the substrate 2, the three kinds of secondary light detection pixels 10, 20 and 30 which selectively sense lights of respectively different wavelength regions (BGR) are sequentially formed and layered, with at least the scaling insulation films 18 and 28 interposed between the secondary light detection pixels that are adjacent in the thickness direction. Further, when the secondary light detection pixels 10, 20 and 30 are being formed, the photoelectric conversion portions 14, 24 and 34, which photoelectrically convert the lights that arc to be sensed, and the signal output portions 12, 22 and 32, which output signals from the field effect thin film transistors 40 in accordance with charges produced by the photoelectric conversion portions 14, 24 and 34, are formed. Here, each field effect thin film transistor 40 includes the gate electrode 44, the gate insulation film 46, the semiconductor layer 48, the source electrode 50 and the drain electrode 52, and the semiconductor layer 48 is formed of an oxide semiconductor or an organic semiconductor. Therefore, as shown in FIG. 2, the imaging device 1 can be provided in which the primary light detection pixels 4 are arrayed in planar directions of the substrate 2, and the primary light detection pixels 4 are structured by layering the three different kinds of secondary light detection pixels 10, 20 and 30.

A capsule endoscope of a first aspect of the present invention includes at least an imaging device that images a location of a subject and an optical system that focuses the location at the imaging device. The imaging device has plural pixel portions that are arranged in in-plane directions on a substrate. The each pixel portion has a photoelectric conversion portion that includes a lower electrode, a photoelectric conversion layer formed over the lower electrode, and an upper electrode formed over the photoelectric conversion layer; and a signal output portion that outputs a signal based on a charge generated at the photoelectric conversion layer through a field effect thin film transistor that includes at least a gate electrode, a gate insulation film, a semiconductor layer, a source electrode and a drain electrode. The photoelectric conversion portion and the signal output portion are superposed in plan view.

In the capsule endoscope described above, in plan view, the signal output section and the photoelectric conversion portion are superposed in the pixel portion of the imaging device. Therefore, in comparison with, for example, a structure in which a signal portion and a photoelectric conversion portion are not superposed, a projected area of the pixel portion in plan view can be made smaller. Consequently, the imaging device in which the pixel portions are arranged in in-plane directions on the substrate is reduced in size, and as a result, the capsule endoscope incorporating the imaging device is reduced in size.

In the structure of the capsule endoscope of the above-described first aspect, the photoelectric conversion layer may be formed with an organic material.

In the capsule endoscope described above, because the photoelectric conversion layer is formed of an organic material, light permeation of the photoelectric conversion layer is improved.

In the structure of the capsule endoscope of the above-described first aspect, the semiconductor layer of the field effect thin film transistor may be formed with at least one of an oxide semiconductor or an organic semiconductor.

In the capsule endoscope described above, because the semiconductor layer of the field effect thin film transistor is formed of an oxide semiconductor or an organic semiconductor, light permeation is improved in comparison with, for example, a semiconductor layer formed of amorphous silicon. Furthermore, larger currents flow with low voltages, and thus power consumption is reduced.

In the structure of the capsule endoscope of the above-described first aspect, the photoelectric conversion layer may be formed with an organic material, and the semiconductor layer of the field effect thin film transistor may be formed with at least one of an oxide semiconductor or an organic semiconductor.

In the capsule endoscope described above, the photoelectric conversion layer is formed of an organic material and the semiconductor layer of the field effect thin film transistor is formed of an oxide semiconductor or an organic semiconductor. Therefore, light permeation of the photoelectric conversion layer and light permeation of the semiconductor layer are improved.

In the above-described structures of the capsule endoscope, three kinds of the pixel portion, which detect light corresponding, respectively, to three colors of red (R), green (G) and blue (B), may be layered on the substrate with sealing insulation films interposed therebetween.

In the capsule endoscope described above, the three kinds of pixel portions, which detect the lights respectively corresponding to the three colors red (R), green (G) and blue (B), are layered on the substrate with the sealing insulation films interposed therebetween. Accordingly, signal charges corresponding to light of the respective wavelength regions (RGB) can be respectively outputted Hence, by combining the outputted signals, imaging in full color is possible. That is, a capsule endoscope capable of capturing color images is formed.

Because the three kinds of pixel portions which detect lights respectively corresponding to the three colors red (R), green (G) and blue (B) are layered on the substrate, with the sealing insulation films interposed, a projected area of the imaging device in plan view is not widened. Consequently, size is reduced compared to an imaging device with a structure in which three kinds of pixel portions are laid out in in-plane directions. Therefore, a capsule endoscope capable of capturing color images is reduced in size (i.e., size is not increased even with capture of color images being enabled).

Here, when the photoelectric conversion layer is formed of an organic material and the semiconductor layer of the field effect thin film transistor is formed of an oxide semiconductor or an organic semiconductor, light permeation of both the photoelectric conversion layer and the semiconductor layer are improved (substantial transparency is enabled). Therefore, even though the three kinds of pixel portions which detect lights respectively corresponding to the three colors red (R), green (G) and blue (B) are in a layered structure, imaging with high sensitivity is possible.

In the above-described structures of the capsule endoscope, in a case in which the semiconductor layer of the field effect thin film transistor is the oxide semiconductor, the oxide semiconductor may be an amorphous oxide semiconductor.

In the capsule endoscope described above, because the active layer of the field effect thin film transistor is formed of an amorphous oxide, uniform film formation at a low temperature (for example, room temperature) is possible.

In the above-described structures of the capsule endoscope, the substrate may be a flexible substrate.

In the capsule endoscope described above, the substrate is a flexible substrate. Therefore, because the flexible substrate can be freely deformed by bending or the like, designing flexibility is improved. As a result, this contributes to a reduction in size of the capsule endoscope.

In the above-described structures of the capsule endoscope, the field effect thin film transistor may be provided with the semiconductor layer including at least a resistance layer and an active layer with a greater electrical conductivity than the resistance layer, the active layer is in contact with the gate insulation film, and the resistance layer electrically connects between the active layer and at least one of the source electrode or the drain electrode.

In the capsule endoscope described above, in the ON state of the field effect transistor, in which a voltage is applied to the gate electrode, because the active layer which serves as the channel has a large electrical conductivity, field effect mobility of the transistor is high, and a high ON current is provided. In the OFF state, because the electrical conductivity of the resistance layer is small and resistance of the resistance layer is high, the OFF current can be kept low. Therefore, an ON/OFF comparison characteristic is greatly improved. In other words, a field effect transistor exhibiting high field effect mobility and a high ON/OFF ratio is formed. As a result, imaging is performed with high resolution and high sensitivity. In addition, power consumption is lowered.

In the above-described structure of the capsule endoscope, the field effect thin film transistor may include at least the resistance layer and the active layer in a layered state,

In the capsule endoscope described above, because the resistance layer and the active layer of the field effect thin film transistor are in a layered state, a projected area in plan view can be reduced. Therefore, the imaging device in which the pixel portions are arrayed is reduced in size in plan view, and as a result, the capsule endoscope incorporating the imaging device is reduced in size.

As described above, according to the present invention, there is an excellent effect in that a capsule endoscope can be reduced in size.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The exemplary embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A capsule endoscope comprising:
an imaging device that images a location of a subject; and
an optical system that focuses the location at the imaging device,
wherein the imaging device comprises a plurality of pixel portions that are arranged in in-plane directions on a substrate,
the pixel portions each comprising:
a photoelectric conversion portion that includes a lower electrode, a photoelectric conversion layer formed over the lower electrode, and an upper electrode formed over the photoelectric conversion layer; and
a signal output portion that outputs a signal based on a charge generated at the photoelectric conversion layer through a field effect thin film transistor that includes at least a gate electrode, a gate insulation film, a semiconductor layer, a source electrode and a drain electrode,
wherein the photoelectric conversion portion and the signal output portion are superposed in plan view.

2. The capsule endoscope of claim 1, wherein the photoelectric conversion layer is formed with an organic material.

3. The capsule endoscope of claim 1, wherein the semiconductor layer of the field effect thin film transistor is formed with at least one of an oxide semiconductor or an organic semiconductor.

4. The capsule endoscope of claim 1, wherein the photoelectric conversion layer is formed with an organic material, and the semiconductor layer of the field effect thin film transistor is formed with at least one of an oxide semiconductor or an organic semiconductor.

5. The capsule endoscope of claim 1, wherein three kinds of the pixel portions, which detect tight corresponding, respectively, to three colors of red, green and blue, are layered on the substrate with sealing insulation films interposed therebetween.

6. The capsule endoscope of claim 3, wherein, in a case in which the semiconductor layer of the field effect thin film transistor is formed with the oxide semiconductor, the oxide semiconductor is an amorphous oxide semiconductor.

7. The capsule endoscope of claim 1, wherein the substrate is a flexible substrate.

8. The capsule endoscope of claim 1, wherein the field effect thin film transistor is configured such that:
the semiconductor layer includes at least a resistance layer and an active layer with a greater electrical conductivity than the resistance layer,
the active layer is in contact with the gate insulation film, and
the resistance layer electrically connects between the active layer and at least one of the source electrode or the drain electrode.

9. The capsule endoscope of claim 8, wherein the resistance layer and the active layer are provided in a layered state in the field effect thin film transistor.
